(19) United States
(12) Patent Application Publication
(10) Pub. No.: US 2011/0146683 A1
(43) Pub. Date: Jun. 23, 2011

(54) Sensor Model

(75) Inventors: Mehdi M. Jafari, Laguna Hills, CA (US); Rhomere S. Jimenez, San Diego, CA (US); Jeffrey K. Aviano, Escondido, CA (US); Russell P. Rush, Santee, CA (US); Edward R. McCoy, Vista, CA (US); Gail F. Upham, Fallbrook, CA (US)

(73) Assignee: Nellcor Puritan Bennett LLC, Boulder, CO (US)

(21) Appl. No.: 12/713,483

(22) Filed: Feb. 26, 2010

Related U.S. Application Data
(63) Continuation-in-part of application No. 12/643,083, filed on Dec. 21, 2009.

(51) Int. Cl. A61M 16/00; G06F 17/11

(52) U.S. Cl. 128/204.21; 703/2

(57) ABSTRACT

The disclosure describes a novel approach of utilizing a model-based approach for estimating a parameter at the wye without utilizing a sensor at the wye in the circuit proximal to the patient.

### RELATED APPLICATIONS

This application is a continuation-in-part of prior application Ser. No. 12/643,083, filed Dec. 21, 2009, and entitled “Adaptive Flow Sensor Model” which application is hereby incorporated herein by reference.

### INTRODUCTION

Medical ventilators may determine when a patient takes a breath in order to synchronize the operation of the ventilator with the natural breathing of the patient. In some instances, detection of the onset of inhalation and/or exhalation may be used to trigger one or more actions on the part of the ventilator. Accurate and timely measurement of patient airway pressure and lung flow in medical ventilators are directly related to maintaining patient-ventilator synchrony and spirometry calculations and pressure-flow-volume visualizations for clinical decision making.

In order to detect the onset of inhalation and/or exhalation, and/or obtain a more accurate measurement of inspiratory and expiratory flow/volume, a flow or pressure sensor may be located close to the patient. For example, to achieve timely non-invasive signal measurements, differential-pressure flow transducers may be placed at the patient wye proximal to the patient. However, the ventilator circuit and particularly the patient wye is a challenging environment to make continuously accurate measurements. The harsh environment for the sensor is caused, at least in part, by the condensations resulting from the passage of humidified gas through the system as well as secretions emanating from the patient. Over time, the condensate material can enter the sensor tubes and/or block its ports and subsequently jeopardize the functioning of the sensor. Additionally, inter-patient cross contamination can occur.

### SUMMARY

The disclosure describes a novel approach of utilizing a model-based approach for estimating a parameter at the wye without utilizing a sensor at the wye.

In part, this disclosure describes a method for estimating at least one parameter at the patient circuit wye in a medical ventilator providing ventilation to a patient. The method includes performing the following steps:

a) monitoring at least one of ventilator settings, internal measurements, available hardware characteristics, and patient characteristics;

b) extracting respiratory mechanics of the patient from ventilator data by fitting a curve based on at least one of the ventilator settings, the internal measurements, the available hardware characteristics, and the patient characteristics, wherein said fitting relies on one or more fit parameters, and wherein the values of said one or more fit parameters are found by said fitting;

(c) calculating a first estimate of at least one parameter at a patient circuit wye for a time interval with at least one sensor model based on at least one of the ventilator settings, the internal measurements, the available hardware characteristics, the patient characteristics, and the one or more fit parameters; and

d) displaying the first estimate of the at least one parameter at the patient circuit wye for the time interval.

Yet another aspect of this disclosure describes a pressure support system that includes: a processor; a pressure generating system adapted to generate a flow of breathing gas controlled by the processor; a housing, the housing contains at least one of the processor and the pressure generating system; at least one sensor, the at least one sensor located in the housing; a ventilation system comprising a patient circuit controlled by the processor, the patient circuit comprising a wye with an inspiration limb and an expiration limb; a patient interface, the patient interface connected to the patient circuit; and a sensor model in communication with the processor, the sensor model is adapted to estimate at least one parameter at the wye based on at least one reading from the at least one sensor in the housing.

In yet another aspect, the disclosure describes a medical ventilator system that includes: a processor; a patient circuit, the patient circuit comprising a wye with an inspiration limb and an expiration limb; a patient interface, the patient interface connected to the patient circuit; a gas regulator controlled by the processor, the gas regulator adapted to regulate a flow of gas from a gas supply to a patient via the patient circuit; a ventilator housing, the ventilator housing contains at least one of the processor and the gas regulator; at least one sensor, the at least one sensor located in the ventilator housing; and a sensor model in communication with the processor, the sensor model is adapted to estimate at least one parameter at the wye based on at least one reading from the at least one sensor during ventilation of a patient by the medical ventilator.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments systems and methods described below and are not meant to limit the scope of the invention in any manner, which scope shall be based on the claims appended hereto.

FIG. 1 illustrates an embodiment of a ventilator connected to a human patient.

FIG. 2 illustrates an embodiment of a ventilator with a proximal sensor model.

FIG. 3 illustrates an embodiment of a method for estimating at least one parameter at the patient circuit wye in a medical ventilator providing ventilation to a patient.

FIG. 4 illustrates an embodiment of a method for estimating at least one parameter at the patient circuit wye in a medical ventilator providing ventilation to a patient.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. The reader will understand that the technology described in the context of a medical ventilator for human patients could be adapted for use with other systems such as ventilators for non-human patients and general gas transport systems in which provide for harsh sensor environments.

Medical ventilators are used to provide a breathing gas to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gas having a desired concentration of oxygen is supplied to the patient at desired pressures and rates. Ventilators capable of operating independently of external sources of pressurized air are also available.

While operating a ventilator, it is desirable to monitor the rate at which breathing gas is supplied to the patient. Some systems have interposed flow and/or pressure sensors at the patient wye proximal to the patient. However, the ventilator circuit and particularly the patient wye is a challenging environment to make continuously accurate measurements. The harsh environment for the sensor is caused by condensation resulting from the passage of humidified gas through the system as well as secretion emanating from the patient. Over time, the condensate material can enter the sensor tubing and/or block its ports and subsequently jeopardize the functioning of the transducer. In addition, the risk of inter-patient cross contamination has to be addresses.

To avoid maintenance issues and costs related to the use and operation of an actual proximal flow sensor with its accompanying electronic and pneumatic hardware, a proximal sensor model (virtual sensor or virtual sensor model) may be utilized to estimate parameters such as proximal wye pressure and flow in a sensorless fashion. The values for the model parameters can be dynamically updated based on ventilator settings, internal measurement, available hardware characteristics, and/or patient's respiratory mechanics parameters extracted from ventilatory data.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software or firmware components described herein as would be understood by those skilled in the art now and hereafter.

As discussed above, proximal sensors have hardware costs and operational issues. For instance the sensors may be blocked from sending patient data during ventilation causing patient data gaps. However, the proximal sensor model (virtual sensor or virtual sensor model) estimates patient data, such as flow rate and pressure, in the patient circuit proximal to the patient or at the wye without the hardware costs or operational issues that are associated with a physical sensor. These estimates are saved, sent, and/or displayed by the ventilator and provide comparable information as obtained by a physical sensor. These estimates provide care-givers, patients, and the ventilators with continuously available information and allow for more informed patient treatment and diagnoses. In an embodiment, the proximal flow and pressure at patient circuit wye are estimated by utilizing at least one of ventilator settings, internal measurements, available hardware characteristics, and patient's respiratory mechanics parameters extracted from ventilatory data versus time in a fitting curve.

In an embodiment, a virtual sensor model (or a bank of multiple models) of a sensor at the patient wye is designed and trained (values assigned to model parameters) to represent dynamics of the patient-ventilator system relevant to estimation of parameters of interest (e.g., flow, pressure). Further, in yet another embodiment, the model uses as inputs parameters based on the one or more fit parameters and at least one of the ventilator settings, the internal measurements, the available hardware characteristics, and the patient characteristics to provide sensor estimates of parameters at the wye as an output.

In one embodiment, the proximal flow and pressure at patient circuit wye are estimated by utilizing the following model equations:

P_{y}(t)=Pₑₓₕ(t)+Q_{c}(t)*(K₁+K₂*Q_{c}(t)); and

Q_{c}(t)=Qₑₓₕ(t)+C_{ef}*Pₑ(t).

Wherein:

P_{y}=pressure at patient circuit wye extracted from ventilator data and circuit characteristics obtained through the ventilator calibration Self-Test process;

Q_{c}=flow rate in the exhalation limb, which is derived or calculated utilizing the above equation;

C_{ef}=compliance of exhalation filter and is a determined constant;

K₁, K₂=parameters of exhalation circuit limb resistance and are modeling parameters for the flow going through the circuit;

Pₑₓₕ=pressure at the exhalation port extracted from ventilator data;

Qₑₓₕ=flow at exhalation port extracted from ventilator data;

t=a continuous variable and stands for time in seconds as it elapses;

P_{y}(t)=the wye pressure estimate at time t; and

Pₑ=conditioned (filtered) time domain derivative of pressure (rate of change of pressure with time) measured at exhalation port, this slope may be calculated utilizing the following model equations in the frequency domain:

$${\dot{P}}_{e} ( s ) = \frac{s}{( s + {p}_{1} ) ( s + {p}_{2} ) ( β s + 1 )} {P}_{e} ( s ) ;$$

$${Q}_{y} ( s ) = {T}_{1} ( s ) * {Q}_{v} ( s ) + {T}_{2} ( s ) * {P}_{y} ( s ) + {E}_{Qy} ( s ) ;$$

Pₑ=pressure at the exhalation port extracted from ventilator;

Q_{y}(t)=estimated proximal flow at the patient circuit wye;

Qᵥ(t)=Q_{del}(t)−Qₑₓₕ(t);

Q_{del}(t)=total flow delivered by the ventilator;

E_{Qy}(t)=approximation residual or estimation error;

Q_{y}(s)=Laplace transform of the flow rate at the patient circuit wye;

T₁(s)Qᵥ(s)=the Laplace transform of the contribution of the ventilator flow rate to the patient flow rate;

T₂(s)*P_{y}(s)=the Laplace transform of the contribution of pressure at patient circuit wye to patient flow rate;

$${T}_{1} ( s ) = d \frac{s + {z}_{1}}{( s + {p}_{3} ) ( s + {p}_{4} )} ; and$$

$${T}_{2} ( s ) = - m * {T}_{1} ( s ) * \frac{s}{( s + {p}_{5} ) ( s + {p}_{6} )} .$$

s=Laplace variable;

z, p₁, p₂, p₃, p₄, p₅, and p₆=model parameters representing system dynamics

β=filtering parameter; and

d and m=modeling parameters.

Pₑ is used in the calculation of Q_{c} and P_{y} for Q_{y} estimation. The model parameters are dynamically updated based on ventilator settings, internal measurements (pressure, flow, etc.), available hardware characteristics, and estimated parameters of patient's respiratory mechanics extracted from ventilatory data. Additionally, one or more of these parameters may assume different values depending on the breath phase (inhalation or exhalation).

The model described above is but one example of how an estimate may be obtained based on the current settings and readings of the ventilator. Alternative model parameters and more involved modeling strategies (building a bank of models to serve different ventilator settings and/or patient conditions) may also be utilized. Furthermore, other wave-shaping modeling approaches and waveform quantifications and modeling techniques may be utilized for hardware and/or respiratory parameter characterization. Furthermore, parameters of such models may be dynamically updated and optimized during ventilation.

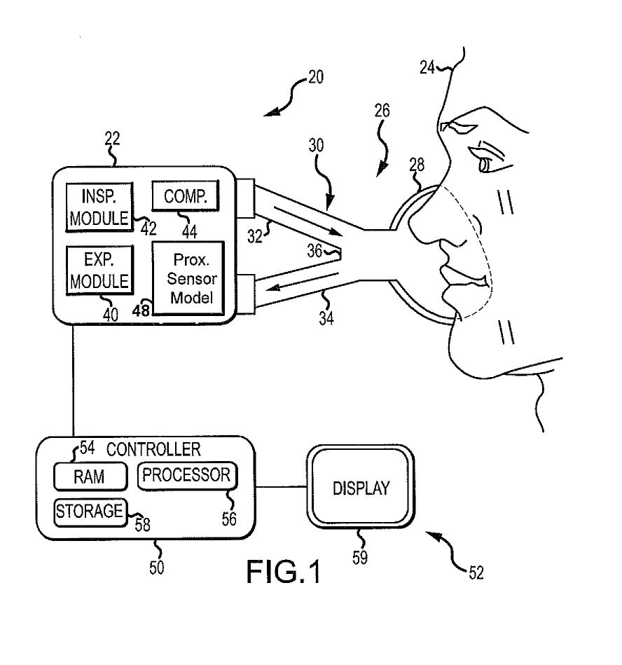

FIG. 1 illustrates an embodiment of a ventilator 20 connected to a human patient 24. Ventilator 20 includes a pneumatic system 22 (also referred to as a pressure generating system 22) for circulating breathing gases to and from patient 24 via the ventilation tubing system 26, which couples the patient 24 to the pneumatic system 22 via physical patient interface 28 and ventilator circuit 30. Ventilator circuit 30 could be a two-limb or one-limb circuit for carrying gas to and from the patient 24. In a two-limb embodiment as shown, a wye fitting 36 may be provided as shown to couple the patient interface 28 to the inspiratory limb 32 and the expiratory limb 34 of the circuit 30.

The present systems and methods have proved particularly advantageous in invasive settings, such as with endotracheal tubes. The present description contemplates that the patient interface 28 may be invasive or non-invasive, and of any configuration suitable for communicating a flow of breathing gas from the patient circuit to an airway of the patient 24. Examples of suitable patient interface devices include a nasal mask, nasal/oral mask (which is shown in FIG. 1), nasal prong, full-face mask, tracheal tube, endotracheal tube, nasal pillow, etc.

Pneumatic system 22 may be configured in a variety of ways. In the present example, system 22 includes an expiratory module 40 coupled with an expiratory limb 34 and an inspiratory module 42 coupled with an inspiratory limb 32. Compressor 44 or another source or sources of pressurized gas (e.g., pressured air and/or oxygen controlled through the use of one or more gas regulators) is coupled with inspiratory module 42 to provide a source of pressurized breathing gas for ventilatory support via inspiratory limb 32.

The pneumatic system 22 may include a variety of other components, including sources for pressurized air and/or oxygen, mixing modules, valves, sensors, tubing, accumulators, filters, etc. Controller 50 is operatively coupled with pneumatic system 22, signal measurement and acquisition systems, and an operator interface 52 may be provided to enable an operator to interact with the ventilator 20 (e.g., change ventilator settings, select operational modes, view monitored parameters, etc.). Controller 50 may include memory 54, one or more processors 56, storage 58, and/or other components of the type commonly found in command and control computing devices.

The memory 54 is non-transitory computer-readable storage media that stores software that is executed by the processor 56 and which controls the operation of the ventilator 20. In an embodiment, the memory 54 comprises one or more solid-state storage devices such as flash memory chips. In an alternative embodiment, the memory 54 may be mass storage connected to the processor 56 through a mass storage controller (not shown) and a communications bus (not shown). Although the description of non-transitory computer-readable media contained herein refers to a solid-state storage, it should be appreciated by those skilled in the art that non-transitory computer-readable storage media can be any available media that can be accessed by the processor 56. Non-transitory computer-readable storage media includes volatile and non-volatile, removable and non-removable media implemented in any method or technology for storage of information such as computer-readable instructions, data structures, program modules or other data. Non-transitory computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the processor 56.

As described in more detail below, controller 50 issues commands to pneumatic system 22 in order to control the breathing assistance provided to the patient 24 by the ventilator 20. The specific commands may be based on inputs received from patient 24, pneumatic system 22 and sensors, operator interface 52 and/or other components of the ventilator 20. In the depicted example, operator interface 52 includes a display 59 that is touch-sensitive, enabling the display 59 to serve both as an input user interface and an output device.

The ventilator 20 is also illustrated as having a virtual proximal sensor model (the “Prox. Sensor Model” in FIG. 1) 48 in pneumatic system 22. The proximal sensor model 48 estimates at least one parameter, such as flow rate and pressure, proximal to the patient 24 in the patient circuit, such as at the wye.

Further, in the embodiment shown, the controller 50 utilizes the ongoing ventilator measurements taken by the ventilator 20 and the ventilator settings in the proximal sensor model 48 to simulate at least one parameter at the patient circuit wye during ventilation. The proximal sensor model 48 may be based on inputs received from patient 24, pneumatic system 22, sensors, and operator interface 52 and/or other components of the ventilator 20. The proximal sensor model 48 can be stored in and utilized by the controller 50, by a computer system located in the ventilator 20, or by an independent source that is operatively coupled with the pneumatic system 22 or ventilator 20.

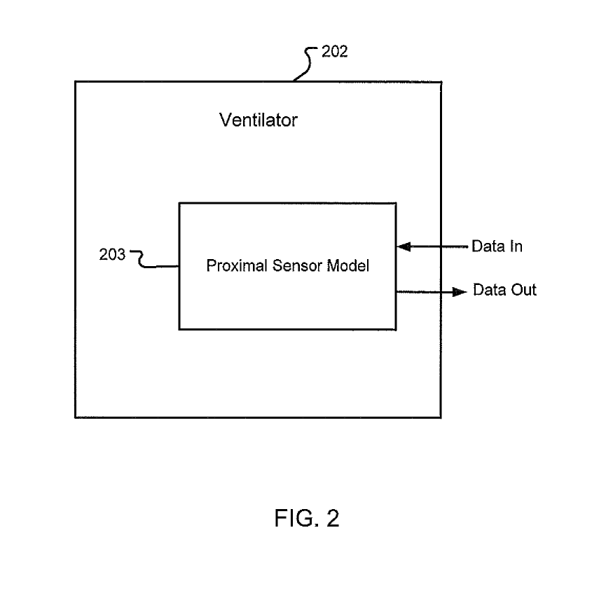

The proximal sensor model 48 may also interact with the signal measurement and acquisition systems, the controller 50 and the operator interface 52 to enable an operator to interact with the model 48, the model 48, the ventilator 20, and the display 59. Further, this coupling allows the controller to receive and display the estimated patient sensor readings produced by the proximal sensor model 48. This computer system may include memory, one or more processors, storage, and/or other components of the type commonly found in command and control computing devices. Furthermore, a proximal sensor model 48 may be integrated into the ventilator 20 as shown, or may be a completely independent component residing on an external device (such as another computing system). The proximal sensor model 48 and its functions are discussed in greater detail with reference to FIG. 2.

FIG. 2 illustrates an embodiment of a ventilator 202 that includes a proximal sensor model 203. The proximal sensor model 203 may be implemented as an independent, stand-alone module, e.g., as a separate software routine either inside the ventilator 203 or within a separate device with data acquisition and transmission as well as computing capabilities connected to or in communication with the ventilator 202. Alternatively, the proximal sensor model 203 may be integrated with software of firmware of the ventilator 202 or another device, e.g., built into a ventilator control board.

As discussed above, a physical sensor at the wye circuit has hardware costs and may have additional maintenance issues. The sensor model 203 estimates patient data during ventilation without a sensor. These estimates are saved, sent, and/or displayed in the ventilator eliminating gaps in patient sensor data. These estimates provide care-givers, patients, and the ventilators with more comprehensive information and allow for more informed patient treatment and diagnoses.

The proximal sensor model 203 may be controlled by any suitable component, such as the ventilator controller, and a separate microprocessor. In this embodiment, the proximal sensor model 203 includes a microprocessor executing software stored either on memory within the processor or in a separate memory cache. The proximal sensor model 203 transmits the estimated sensor data to other devices or components of the ventilator.

As discussed above, the controller may also interface between the ventilator and the proximal sensor model 203 to provide information such as data pertaining to system dynamics and/or previous ventilator settings, internal measurements, available hardware characteristics, and patient's respiratory mechanics parameters extracted from ventilator data. In one embodiment, the ventilator settings include circuit type and its characteristics (resistance and compliance), humidification system data, interface type and size, breath type, breath delivery parameters such as tidal volume, target pressure, end positive expiratory pressure (PEEP), and/or oxygen mix. This list is not limiting, Any suitable ventilator setting may be utilized by the proximal sensor model 203. In another embodiment, the internal measurements include delivered and exhausted flow rates, pressure measurements at the inhalation and exhalation manifolds, breath phase (inhalation, exhalation), gas temperature, relative humidity, and atmospheric pressure. This list is not limiting. Any suitable internal measurement may be utilized by the proximal sensor model 203. In a further embodiment, the available hardware characteristics include patient circuit model parameters, interface model parameters (e.g., endotracheal tube size), humidification system model parameters, and/or gas delivery and exhaust (exhalation subsystem for PEEP control) characteristics. This list is not limiting. Any suitable hardware characteristics may be utilized by the proximal sensor model 203. In another embodiment, the respiratory mechanic parameters include components of patient's respiratory resistance and compliance, patient disease status, and/or other patient characteristics such as age, gender, and weight. This list is not limiting. Any suitable respiratory mechanic parameters may be utilized by the proximal sensor model 203. Further, in one embodiment, the respiratory mechanics are extracted from ventilator data, such as flow and pressure measurements during breath delivery and/or data acquired through execution of specific respiratory maneuvers. This list is not limiting. Any suitable respiratory mechanics may be extracted from ventilator data and utilized by the proximal sensor model 203.

A ventilator controller or a separate controller hosting the virtual sensor model 203 may update information continuously in order to obtain accurate sensor estimates. The ventilator controller or a separate controller hosting the virtual sensor model 203 may also receive information from external sources such as modules of the ventilator, in particular information concerning the current breathing phase of the patient, ventilator parameters and/or other ventilator readings. The received information may include user-selected or predetermined values for various parameters such as tubing parameters, respiratory mechanics, and/or gas conditions (e.g. mix, humidity, and/or temperature). This list is not limiting. Any suitable user-selected or predetermined values for parameters may be extracted from ventilator data and utilized by the proximal sensor model 203. The received information may further include reset commands, criteria for model selection, and/or execution of a calibration or model training maneuver. This list is not limiting. Any suitable received information may be utilized by the proximal sensor model 203. The controller or a separate controller hosting the virtual sensor model 203 may also include an internal timer so that individual patient sensor data estimates can be performed at a user or manufacturer specified interval.

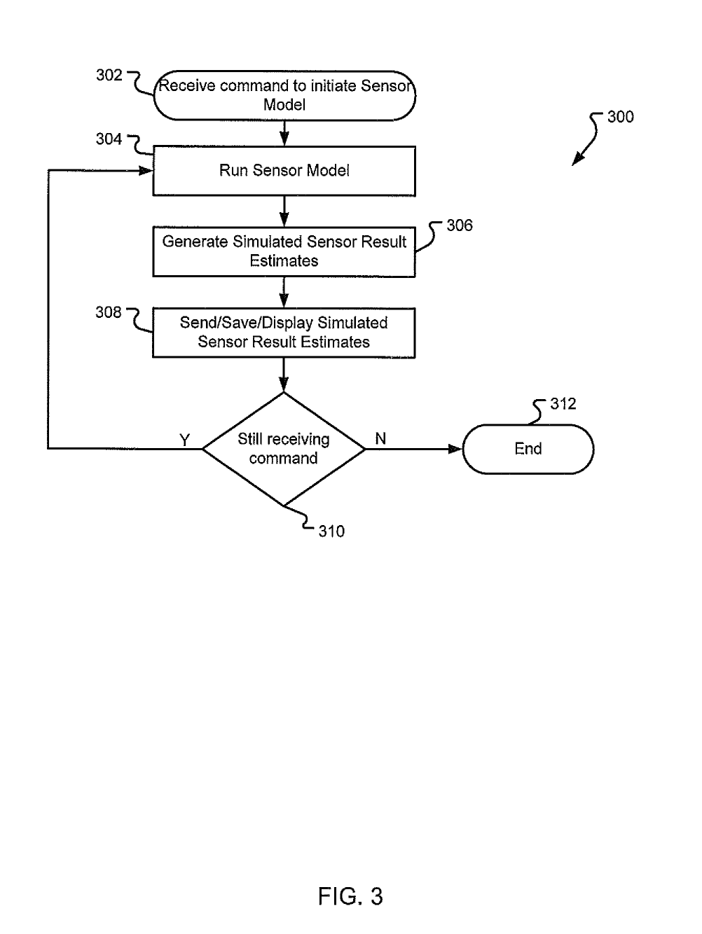

FIG. 3 represents an embodiment of a method for estimating at least one parameter at the patient circuit wye in a medical ventilator providing ventilation to a patient, 300.

As illustrated, method 300 receives a command to initiate a sensor model, 302. In one embodiment, the command is from a controller, such as a pressure support system controller, a sensor model controller, or a ventilator controller. In an alternative embodiment, the command is inputted by a user through a user interface. In another embodiment, the command is configured into the ventilator.

In response to this command, method 300 runs the sensor model, 304 and generates simulated sensor result estimates, 306. In one embodiment, the model utilizes current and/or past ventilator settings, internal measurements, available hardware characteristics, and patient's respiratory mechanics parameters extracted from ventilator data to generate the simulated sensor result estimates. In one embodiment, the estimates are flow rate and/or pressure. The model for the system may be any suitable model as long as it can provide a reasonably accurate prediction of the pressure and/or flow at the wye based on past patient circuit wye estimates and current and/or past ventilator sensor readings. In one embodiment, the model equations (in time and frequency domains) for the modeling process are:

$${P}_{y} ( t ) = {P}_{exh} ( t ) + {Q}_{c} ( t ) * ( {K}_{1} + {K}_{2} * {Q}_{c} ( t ) ) ;$$

$${Q}_{c} ( t ) = {Q}_{exh} ( t ) + {C}_{ef} * {\dot{P}}_{e} ( t ) ;$$

$${\dot{P}}_{e} ( s ) = \frac{s}{( s + {p}_{1} ) ( s + {p}_{2} ) ( β s + 1 )} {P}_{e} ( s ) ;$$

$${Q}_{y} ( s ) = {T}_{1} ( s ) * {Q}_{v} ( s ) + {T}_{2} ( s ) * {P}_{y} ( s ) + {E}_{Qy} ( s ) ;$$

$${T}_{1} ( s ) = d \frac{s + {z}_{1}}{( s + {p}_{3} ) ( s + {p}_{4} )} ; and$$

$${T}_{2} ( s ) = - m * {T}_{1} ( s ) * \frac{s}{( s + {p}_{5} ) ( s + {p}_{6} )} .$$

Next, method 300 sends, saves, and/or displays these estimates, 308. In one embodiment, the estimates are sent to a display and listed upon the display. In an embodiment, the estimates are sent to a controller. The controller may utilize the estimates to control other ventilator components or to adjust the sensor model. In another embodiment, the estimates are sent from the memory to a display based on an inputted user command or pre-set command.

Method 300 includes a first determination operation 310 that determines if a command is still being received. Upon determination that a command is being received, method 300 repeats the running of the sensor model, 304. Upon determination that a command is not being received, method 300 ends, 312. In an embodiment, the duration of the command is a pre-set time interval entered by a user and/or programmed into the ventilator.

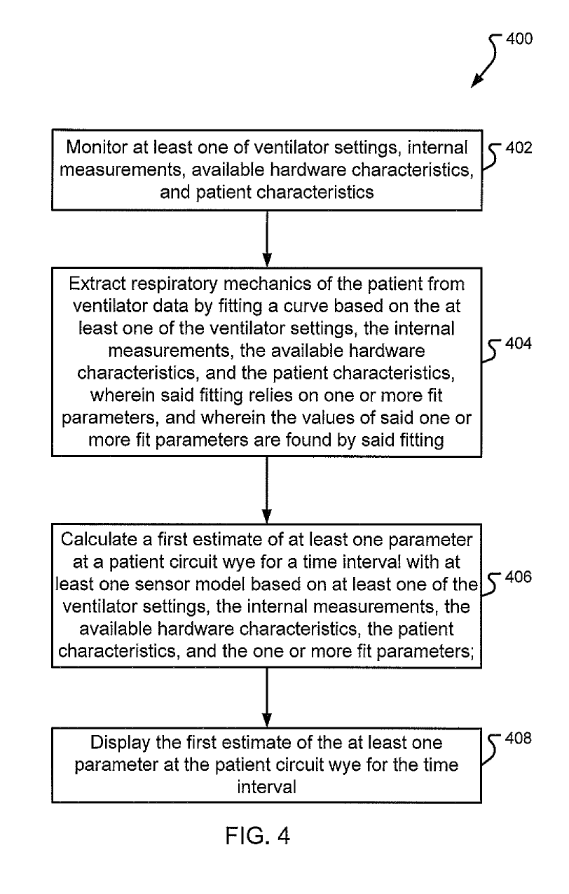

FIG. 4 represents an embodiment of a method for estimating at least one parameter at the patient circuit wye in a medical ventilator providing ventilation to a patient, 400.

As illustrated, method 400 monitors at least one of ventilator settings, internal measurements, available hardware characteristics, and patient characteristics (e.g. patient's respiratory mechanics parameters extracted from ventilatory data) 402. In one embodiment, the ventilator settings include circuit type and its characteristics (resistance and compliance), humidification system data, interface type and size, breath type, and/or breath delivery parameters such as tidal volume, target pressure, end positive expiratory pressure (PEEP), and/or oxygen mix. This list is not limiting. Any suitable ventilator setting may be utilized by method 400. In another embodiment, the internal measurements include delivered and exhausted flow rates, pressure measurements at the inhalation and exhalation manifolds, breath phase (inhalation, exhalation), gas temperature, relative humidity, and/or atmospheric pressure. This list is not limiting. Any suitable internal measurement may be utilized by method 400. In a further embodiment, the available hardware characteristics include patient circuit model parameters, interface model parameters (e.g., endotracheal tube size), humidification system model parameters, and/or gas delivery and exhaust (exhalation subsystem for PEEP control) characteristics. This list is not limiting. Any suitable hardware characteristics may be utilized by 400.

Further, method 400 extracts respiratory mechanics of the patient from ventilator data by fitting a curve based on at least one of the ventilator settings, the internal measurements, the available hardware characteristics, and the patient characteristics, wherein said fitting relies on one or more, 404. In another embodiment, the respiratory mechanics of the patient include components of patient's respiratory resistance and compliance, patient disease status, and/or other patient characteristics such as age, gender, and/or weight. This list is not limiting. Any suitable respiratory mechanic parameters may be utilized by method 400. Further, in one embodiment, the respiratory mechanics are extracted from ventilator data, such as flow and pressure measurements during breath delivery and/or data acquired through execution of specific respiratory maneuvers. This list is not limiting. Any suitable respiratory mechanics may be extracted from ventilator data and utilized by method 400. The respiratory mechanics data are extracted by utilizing methods such as a least square curve fitting algorithm applied to breath data or data acquired through execution of a respiratory maneuver.

The model for the curve may be any suitable model as long as it can provide a reasonably accurate prediction of the pressure and/or flow at the wye based on past and/or current ventilator settings, internal measurements, available hardware characteristics, and patient's respiratory mechanics parameters extracted from ventilator data. In one embodiment, the model equations for the fitted curve to estimate respiratory parameters are:

P_{aw}(t)=E∫Qdt+QR−Pₘ(t).

“P_{aw}” in the above equation is pressure measured at the patient interface. “Pₘ” in the above equation is pressure generated by the inspiratory muscles of the patient. Further, “Pₘ” may be used as the index of the patient's effort. “E” in the above equation is lung elastance (which is the inverse of lung compliance, i.e., E=1/C). “Q” in the above equation represents instantaneous lung flow and “R” in the above equation is lung resistance.

The fitting relies on one or more fit parameters. The values of said one or more fit parameters are found by said fitting. The fit parameters may be constants chosen based on the specific patient type, the ventilator application, and other ventilator parameters.

In one embodiment, respiratory parameters and tubing characteristics (such as estimated respiratory compliance, breathing circuit and endotracheal tube resistance and compliance) are used to determine an appropriate virtual sensor model type and/or assign values to model parameters. In one embodiment, such a model would consist of the following equations:

$${P}_{y} ( t ) = {P}_{exh} ( t ) + {Q}_{c} ( t ) * ( {K}_{1} + {K}_{2} * {Q}_{c} ( t ) ) ;$$

$${Q}_{c} ( t ) = {Q}_{exh} ( t ) + {C}_{ef} * {\dot{P}}_{e} ( t ) ;$$

$${\dot{P}}_{e} ( s ) = \frac{s}{( s + {p}_{1} ) ( s + {p}_{2} ) ( β s + 1 )} {P}_{e} ( s ) ;$$

$${Q}_{y} ( s ) = {T}_{1} ( s ) * {Q}_{v} ( s ) + {T}_{2} ( s ) * {P}_{y} ( s ) + {E}_{Qy} ( s ) ;$$

$${T}_{1} ( s ) = d \frac{s + {z}_{1}}{( s + {p}_{3} ) ( s + {p}_{4} )} ; and$$

$${T}_{2} ( s ) = - m * {T}_{1} ( s ) * \frac{s}{( s + {p}_{5} ) ( s + {p}_{6} )} .$$

In one embodiment, step 404 includes building a proximal flow sensor model (or a bank of multiple models) to represent dynamics of the patient-ventilator system relevant for estimating at least one parameter, such as flow rate and/or pressure, at the patient wye. The model uses as inputs parameters based on at least one of the one or more fit parameters, the at least one of the ventilator settings, the internal measurements, the available hardware characteristics, and the patient characteristics.

Method 400 calculates a first estimate of at least one parameter at a patient circuit wye for a time interval with at least one sensor model based on at least one of the ventilator settings, the internal measurements, the available hardware characteristics, the patient characteristics, and the one or more fit parameters, 406. In an embodiment, the time interval is pre-set time entered by a user into the ventilator. In an additional embodiment, the time interval is programmed or configured into the ventilator. In one embodiment, the first estimate of the at least one parameter at the patient circuit wye is pressure. In an additional embodiment, the first estimate of the at least one parameter at the patient circuit wye is flow rate.

The estimate of the first estimate of the at least one parameter at the patient circuit wye for the time interval is displayed by method 400, 408. The displaying step, 408 of method 400 may further include displaying the first estimate of the at least one parameter at the patient circuit wye for the time interval when the at least one of the ventilator settings, the internal measurements, the available hardware characteristics, and the patient characteristics have a predetermined value. In an alternative embodiment, the displaying step, 408 of method 400 includes displaying the first estimate of the at least one parameter at the patient circuit wye for the time interval only when the at least one of the ventilator settings, the internal measurements, the available hardware characteristics, and the patient characteristics or patient's respiratory mechanics parameters extracted from ventilatory data. In one embodiment, the displaying step of method 400 includes displaying the first estimate of the at least one parameter at the patient circuit wye for the time interval when the ventilator is performing a predetermined action.

In yet another embodiment, model selection and/or values assigned to model parameters are optimized on a regressive basis over one or several breaths using physical laws of conservation logic and causality to modify model parameters. Examples of such accuracy checking mechanisms include but are not limited to volume balance. The volume balance may be utilized for a cyclical behavior like respiration. Net volume input and output from a closed system without leakage may integrate to null over one or a multiple of complete duty cycles. Further, in a ventilator tubing system with gas flow moving from upstream (inhalation manifold) to downstream (exhalation manifold), the mid stream pressure (circuit wye) may not exceed upstream pressure or be less than downstream pressure. In another example, the total volume delivered to the lungs during inhalation may not exceed the total volume entering patient circuit at the ventilator output. In one embodiment, lung flow and airway pressure are estimated by the virtual sensor model and used to derive lung mechanic parameters. Theses parameters may then be compared to the values provided by the operator or estimates derived from ventilator data or obtained through implementation of specific respiratory maneuvers.

### Example

The following equations express the current discretized implementation of the NPB 840 ventilator for the neonatal patient setting. The variable “n” is equal to interval of measurement. In one embodiment, “n” is used to count discrete intervals of 10 or 5 milliseconds (ms) each. The NPB 840 ventilator utilizes a 5 ms sampling interval and characterizes the components of the tubing including patient circuit resistance and compliance. In this implementation, E_{Qy} is assumed negligible.

P_{y}(n)=Pₑₓₕ(n)+Q_{c}(n)*(K₁+K₂*Q_{c}(n));

Q_{c}(n)=Qₑₓₕ(n)+C_{ef}*\dot{P}ₑ(n);

\dot{P}ₑ(n)=0.185*(P_{fe}(n)−P_{fe}(n−1))+0.0745*\dot{P}ₑ(n−1)−0.000023*\dot{P}ₑ(n−2)

P_{fe}(n)=0.65*(P_{fe}(n−1)+0.35*Pₑ(n); P_{fe}(0)=0.0

\dot{P}_{y}(n)=0.043*((P_{y}(n)−P_{y}(n−1))+0.8714*\dot{P}_{y}(n−1)−0.0884*\dot{P}_{y}(n−2)

Q₁(n)=Qᵥ(n)−m*\dot{P}_{y}(n)

Q₂(n)=g₁*Q₂(n−1)+g₂*Q₁(n)

Q_{y}(n)=A1*Qᵥ(n−1)+A2*Q₂(n)−A3*Q₂(n−1)

$$A 1 = \frac{1}{1 + 0.005 * c}$$

$$A 2 = \frac{a * ( 1 + 0.005 * b )}{1 + 0.005 * c}$$

$$A 3 = \frac{a}{1 + 0.005 * c}$$

Model parameters a, b, c, g₁, g₂, and m are dynamically updated based on ventilator settings, internal measurements (pressure, flow, etc.), available hardware characteristics (circuit resistance and compliance, endotracheal tube size), and patient's respiratory mechanics parameters extracted from ventilatory data. Additionally, one or more of these parameters may assume different values depending on the breath phase (inhalation or exhalation). In this example for neonatal patients, b, and c were fixed as follows: b=2.0; c=2.5. The interim variable “cest” was computed and used in conjunction with the endotracheal tube size to extract values for “a”, “m”, g₁, g₂, from lookup tables using interpolation for in-between index entries.

$$cest = \frac{0.5 * ( {V}_{te} + {V}_{ti} )}{[ ( {P}_{iend} - {P}_{eend} ) - ( {K}_{1} * {Q}_{iend} + {K}_{2} * {Q}_{eend} * {Q}_{eend} ) ]}$$

Vₜₑ=exhaled tidal volume (extracted from ventilator signals, in ml);
Vₜᵢ=inspired tidal volume (extracted from ventilator signals, in ml);
P_{iend}=end inspiratory pressure (extracted from ventilator signals, in cmH2O)
P_{eend}=end expiratory pressure (extracted from ventilator signals, in cmH2O)
Q_{iend}=end inspiratory flow (extracted from ventilator signals, in liters per minute)
Q_{eend}=end expiratory flow (extracted from ventilator signals, in liters per minute)
For example, Table 1 illustrates the parameters of exhalation circuit limb resistance and modeling parameters for the flow going through the circuit for various endotracheal tube sizes for the NPB 840.

TABLE 1

| ETT ID (mm) | K₁ | K₂ |
|---|---|---|
| 2.0 | 1.09 | 0.4519 |
| 2.5 | 0.4869 | 0.1777 |
| 3.0 | 0.2348 | 0.0879 |
| 3.5 | 0.1571 | 0.0491 |

In another example, tables 2A, 2B, 2C, 3, and 4 show the values for “a”, “m”, “g₁”, and “g₂”. An interim variable “cest” is computed in conjunction with the endotracheal tube size to extract “a” and “m” from lookup tables using interpolation for in-between index entries for the NPB 840.

TABLE 2A
“a” values versus cest

| | cest | | | | | | | | |
|---|---|---|---|---|---|---|---|---|---|
| ETT ID (mm) | 0.10 | 0.20 | 0.30 | 0.40 | 0.50 | 0.60 | 0.70 | 0.80 | 0.90 |
| 2.0 | 0.20 | 0.25 | 0.25 | 0.35 | 0.35 | 0.35 | 0.35 | 0.35 | 0.35 |
| 2.5 | 0.20 | 0.30 | 0.30 | 0.40 | 0.40 | 0.40 | 0.50 | 0.50 | 0.50 |
| 3.0 | 0.30 | 0.50 | 0.50 | 0.50 | 0.50 | 0.50 | 0.60 | 0.60 | 0.60 |
| 3.5 | 0.20 | 0.30 | 0.30 | 0.40 | 0.40 | 0.40 | 0.50 | 0.50 | 0.50 |

TABLE 2B
“a” values versus cest

| | cest | | | | | | | | |
|---|---|---|---|---|---|---|---|---|---|
| ETT ID (mm) | 1.00 | 1.10 | 1.20 | 1.30 | 1.40 | 1.50 | 1.60 | 1.70 | 1.80 |
| 2.0 | 0.35 | 0.35 | 0.35 | 0.35 | 0.35 | 0.35 | 0.35 | 0.35 | 0.35 |
| 2.5 | 0.60 | 0.60 | 0.60 | 0.60 | 0.60 | 0.60 | 0.60 | 0.60 | 0.80 |
| 3.0 | 0.70 | 0.70 | 0.70 | 0.80 | 0.80 | 0.80 | 0.80 | 0.80 | 0.80 |
| 3.5 | 0.60 | 0.60 | 0.60 | 0.60 | 0.60 | 0.70 | 0.70 | 0.70 | 0.80 |

TABLE 2C
“a” values versus cest

| | |
|---|---|
| ETT ID | cest |

| (mm) | 1.90 | 2.0 |
|---|---|---|
| 2.0 | 0.35 | 0.35 |
| 2.5 | 0.80 | 0.80 |
| 3.0 | 0.90 | 0.90 |
| 3.5 | 0.80 | 0.90 |

TABLE 3
“m” values versus cest

cest

| ETT ID (mm) | 0.10 | 0.20 | 0.30 | 0.40 | >0.4 |
|---|---|---|---|---|---|
| 2.0 | 25 | 25 | 15 | 10 | 0 |
| 2.5 | 25 | 25 | 15 | 10 | 0 |
| 3.0 | 25 | 25 | 15 | 10 | 5 |
| 3.5 | 25 | 25 | 15 | 10 | 5 |

TABLE 4
“g₁” and “g₂” values

| ETT ID (mm) | g₁ | g₂ |
|---|---|---|
| 2.0 | 0.75 | 0.25 |
| 2.5 | 0.75 | 0.25 |
| 3.0 | 0.90 | 0.10 |
| 3.5 | 0.90 | 0.10 |

This exemplary embodiment is not meant to be limiting. Additional, algorithms may cover different types of breathing behavior and ventilator settings as well as estimate of patient respiratory parameters. Multiple model parameters and more involved optimization strategies can be utilized as suitable for application needs. Additional estimated parameters related to the time-variant respiratory impedance (resistance, elastance, inductance) or a combination of them may be used as inputs to the virtual sensor model. Furthermore, other wave-shaping and modeling approaches and waveform quantification may be utilized. Moreover, parameters of such models may be dynamically updated and optimized during normal ventilator operation to obtain the best estimated results.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous changes may be made which will readily suggest themselves to those skilled in the art and which are encompassed in the spirit of the disclosure and as defined in the appended claims.

What is claimed is:

1. A method for estimating at least one parameter at a patient circuit wye in a medical ventilator providing ventilation to a patient, the method comprising:
monitoring at least one of ventilator settings, internal measurements, available hardware characteristics, and patient characteristics;
extracting respiratory mechanics of the patient from ventilator data by fitting a curve based on at least one of the ventilator settings, the internal measurements, the available hardware characteristics, and the patient characteristics, wherein said fitting relies on one or more fit parameters, and wherein the values of said one or more fit parameters are found by said fitting;
calculating a first estimate of at least one parameter at a patient circuit wye for a time interval with at least one sensor model based on at least one of the ventilator settings, the internal measurements, the available hardware characteristics, the patient characteristics, and the one or more fit parameters; and
displaying the first estimate of the at least one parameter at the patient circuit wye for the time interval.

2. The method of claim 1, wherein displaying further comprising:
displaying the first estimate of the at least one parameter at the patient circuit wye for the time interval when the at least one of the ventilator settings, the internal measurements, the available hardware characteristics, and the patient characteristics have a predetermined value.

3. The method of claim 1 further comprising:
displaying the first estimate of the at least one parameter at the patient circuit wye for the time interval only when the at least one of the ventilator settings, the internal measurements, the available hardware characteristics, and the patient characteristics do not have a predetermined value.

4. The method of claim 1 wherein the first estimate of the at least one parameter at the patient circuit wye estimate is flow rate.

5. The method of claim 1 wherein the first estimate of the at least one parameter at the patient circuit wye estimate is pressure.

6. The method of claim 1 wherein the sensor model utilizes the following equations (in time and frequency domains) for the step of calculating a first estimate of at least one parameter:
$${P}_{y} ( t ) = {P}_{exh} ( t ) + {Q}_{c} ( t ) * ( {K}_{1} + {K}_{2} * {Q}_{c} ( t ) ) ;$$
$${Q}_{c} ( t ) = {Q}_{exh} ( t ) + {C}_{ef} * {\dot{P}}_{e} ( t ) ;$$
$${\dot{P}}_{e} ( s ) = \frac{s}{( s + {p}_{1} ) ( s + {p}_{2} ) ( β s + 1 )} {P}_{e} ( s ) ;$$
$${Q}_{y} ( s ) = {T}_{1} ( s ) * {Q}_{v} ( s ) + {T}_{2} ( s ) * {P}_{y} ( s ) + {E}_{Qy} ( s ) ;$$
$${T}_{1} ( s ) = d \frac{s + {z}_{1}}{( s + {p}_{3} ) ( s + {p}_{4} )} ; and$$
$${T}_{2} ( s ) = - m * {T}_{1} ( s ) * \frac{s}{( s + {p}_{5} ) ( s + {p}_{6} )} .$$

7. A pressure support system comprising:
a processor;
a pressure generating system adapted to generate a flow of breathing gas controlled by the processor;
a housing, the housing contains at least one of the processor and the pressure generating system;
at least one sensor, the at least one sensor located in the housing;
a ventilation system comprising a patient circuit controlled by the processor, the patient circuit comprising a wye with an inspiration limb and an expiration limb;
a patient interface, the patient interface connected to the patient circuit; and
a sensor model in communication with the processor, the sensor model is adapted to estimate at least one parameter at the wye based on at least one reading from the at least one sensor in the housing.

8. The pressure support system of claim 7, wherein the sensor model is controlled by the processor.

9. The pressure support system of claim 7, wherein the sensor model is controlled by a processor in the sensor model.

10. The pressure support system of claim 7, wherein the at least one parameter at the wye is flow rate.

11. The pressure support system of claim 7, wherein the at least one parameter at the wye is pressure.

12. The pressure support system of claim 7, wherein the sensor model is adapted to utilize the following model equations to estimate the at least one parameter at the wye:
$${P}_{y} ( t ) = {P}_{exh} ( t ) + {Q}_{c} ( t ) * ( {K}_{1} + {K}_{2} * {Q}_{c} ( t ) ) ;$$
$${Q}_{c} ( t ) = {Q}_{exh} ( t ) + {C}_{ef} * {\dot{P}}_{e} ( t ) ;$$
$${\dot{P}}_{e} ( s ) = \frac{s}{( s + {p}_{1} ) ( s + {p}_{2} ) ( β s + 1 )} {P}_{e} ( s ) ;$$
$${Q}_{y} ( s ) = {T}_{1} ( s ) * {Q}_{v} ( s ) + {T}_{2} ( s ) * {P}_{y} ( s ) + {E}_{Qy} ( s ) ;$$
$${T}_{1} ( s ) = d \frac{s + {z}_{1}}{( s + {p}_{3} ) ( s + {p}_{4} )} ; and$$
$${T}_{2} ( s ) = - m * {T}_{1} ( s ) * \frac{s}{( s + {p}_{5} ) ( s + {p}_{6} )} .$$

13. The pressure support system of claim 7, further comprising a display controlled by the processor, the display is adapted to display the estimate of the at least one parameter at the wye.

14. A medical ventilator system, comprising:
a processor;
a patient circuit, the patient circuit comprising a wye with an inspiration limb and an expiration limb;
a patient interface, the patient interface connected to the patient circuit;
a gas regulator controlled by the processor, the gas regulator adapted to regulate a flow of gas from a gas supply to a patient via the patient circuit;
a ventilator housing, the ventilator housing contains at least one of the processor and the gas regulator;
at least one sensor, the at least one sensor located in the ventilator housing; and
a sensor model in communication with the processor, the sensor model is adapted to estimate at least one parameter at the wye based on at least one reading from the at least one sensor during ventilation of a patient by the medical ventilator.

15. The medical ventilator system of claim 14, wherein the sensor model is controlled by a processor in the sensor model.

16. The medical ventilator system of claim 14, wherein the sensor model is controlled by the ventilation system.

17. The medical ventilator system of claim 14, wherein the at least one parameter at the wye is flow rate.

18. The medical ventilator system of claim 14, wherein the at least one parameter at the wye is pressure.

19. The medical ventilator system of claim 14, wherein the sensor model is adapted to utilize the following model equations to estimate the parameter at the wye:
$${P}_{y} ( t ) = {P}_{exh} ( t ) + {Q}_{c} ( t ) * ( {K}_{1} + {K}_{2} * {Q}_{c} ( t ) ) ;$$
$${Q}_{c} ( t ) = {Q}_{exh} ( t ) + {C}_{ef} * {\dot{P}}_{e} ( t ) ;$$
$${\dot{P}}_{e} ( s ) = \frac{s}{( s + {p}_{1} ) ( s + {p}_{2} ) ( β s + 1 )} {P}_{e} ( s ) ;$$
$${Q}_{y} ( s ) = {T}_{1} ( s ) * {Q}_{v} ( s ) + {T}_{2} ( s ) * {P}_{y} ( s ) + {E}_{Qy} ( s ) ;$$
$${T}_{1} ( s ) = d \frac{s + {z}_{1}}{( s + {p}_{3} ) ( s + {p}_{4} )} ; and$$
$${T}_{2} ( s ) = - m * {T}_{1} ( s ) * \frac{s}{( s + {p}_{5} ) ( s + {p}_{6} )} .$$

20. The medical ventilator system of claim 14, further comprising a display controlled by the processor, the display is adapted to display the estimate of the at least one parameter at the wye.
